(19) United States
(12) Patent Application Publication
(10) Pub. No.: US 2009/0149815 A1
(43) Pub. Date: Jun. 11, 2009

(54) Medication dosing system based upon patient classification codes

(76) Inventors: Jeffrey S. Kiel, Gainesville, GA (US); H. Greg Thomas, Carrollton, GA (US)

Correspondence Address: Collen A. Beard;Kiel Laboratories, Inc., 2225 Centennial Drive, Gainesville, GA, 30504, US

(21) Appl. No.: 12/316,361

(22) Filed: Dec. 11, 2008

Related U.S. Application Data
(60) Provisional application No. 61/007,071, filed on Dec. 11, 2007.
(60) Provisional application No. 61/123,803, filed on Apr. 11, 2008.

(51) Int. Cl. A61M 5/31; G06Q 50/00; A61J 7/00

(52) U.S. Cl. 604/189; 705/3; 604/78

(57) ABSTRACT

Medication dosing system for one or more medications based on patient classification codes and standardized dosage units, where the patient classification codes are correlated with one or more characteristics of the patient, such as weight or age, and each medication is formulated so that the dosage unit for the classification code contains the recommended amount of medication for patients within that classification code.

### RELATED APPLICATIONS

The present application is related to and claims priority to U. S. Provisional Application Ser. No. 61/007,071, filed Dec. 11, 2007, and U.S. Provisional Application Ser. No. 61/123,803, filed Apr. 11, 2008, the entire contents of which are incorporated herein by reference.

### BACKGROUND OF THE INVENTION

The invention relates to a medication dosing system. Incorrect medication dosing causes a large number of adverse events each year. These errors can result from improperly provided or deciphered physician prescriptions as well as pharmacist, parent, or caregiver mistakes. Medications are often provided with confusing instructions and with a dispensing device having multiple gradations that increases confusion.

Many studies have been published describing efforts to reduce medication dosing error and many initiatives have resulted, such as computerized physician order entry and decision support systems, some of which have led to a substantial reduction of errors. Color coded dosing systems are described in U.S. Pat. No. 4,926,885 to Hinkle and U.S. Pat. No. 6,132,416 to Broselow. Each of these patents describes a system for dosing of medication or selection of medical equipment based upon an anatomical dimension (e.g. length) of the patient. The medication or medical equipment is color coded and matched to a certain dimension. For example, a child is laid upon a strip having color coded regions and the color of the region corresponding to the length of the child is noted. The caregiver then selects a piece of equipment or a package of medication that corresponds to that color. Each medication is prepackaged in packages of various colors, corresponding to different dosages.

In other embodiments, an automated dispensing system is used to deliver the recommended dosage for a certain inputted color code or an oral syringe having multiple gradations indicated in color is used for dispensing.

When researchers compared the color coded method of Broselow with conventional pediatric dosing methods, they found that the concept of color coded dosing zones has a potential to enhance error detection and reduction. When the color coding method developed by Broselow was compared with standard dosing methods it was found to decrease errors in both the hospital and home environment. However, these color coding methods are not optimal systems and confusion can still arise and lead to dosing errors and adverse events. For example, a child may be given 2 ml of one medication but 4 ml of another medication, each in a red dispensing oral syringe, causing confusion. Oral syringes with multiple gradations in different colors may lead to confusion, particularly in households with more than one child.

Accordingly, there is a need for a medication dosing system that offers consistency and reliability, leading to fewer dosage errors and adverse events.

### SUMMARY OF THE INVENTION

The invention is a medication dosing system for one or more medications based on the use of patient classification codes and standardized dosage units. The patient classification codes are correlated with one or more characteristics of the patient, such as weight or age. Each medication is formulated so that the dosage unit for the classification code contains the recommended amount of medication for patients within that classification code. The patient thus receives the same dosage unit (e.g. 3 ml) of every medication and this dosage unit is determined based upon the classification code assigned to the patient.

The invention is also a medication dosing kit for each classification code that includes the medication and a means for delivering the dosage unit for that classification code.

An object of the invention is to provide a dosing system and dosing kits which reduce the possibility of incorrect medication dosing.

### DETAILED DESCRIPTION OF THE INVENTION

The medication dosing system includes the one or more medications, a patient classification code, a dosage unit assigned to each classification code, and a single dose dispenser customized for each dosage unit.

A dosing kit for each patient classification code includes the medication packaged as single dosage units (such as single tablets) or in bulk, and a single dose dispenser customized for the dosage unit if the medication is packaged in bulk. In addition, a physician information sheet is desirably provided for each medication detailing the patient classification codes, the classification parameters, and the quantities of medication in each dosage unit. The physician information sheet may also provide additional information that might be relevant such as the Clarks Weight Rule information for the medication. A patient information sheet is also desirably included with each kit.

The physician information sheet allows the physician to assign a patient classification code to the patient based upon the classification parameter(s). The code then determines the dosage unit for the patient. The selection of the proper dosage unit is independent of the medication since each medication will have the same dosage unit for the dosing classification code.

Definitions

The term “medication” means any composition that is administered in a regimented manner, including but not limited to pharmaceuticals, medical foods, and dietary supplements.

“Patient classification code” means an arbitrary code that is correlated with the classification parameter.

“Classification parameter” means one or more characteristics of a patient such as weight, height, age, sex, and body surface area.

The term “dosage unit” means a certain quantity of a dosage form. The dosage form can be a liquid, solid, suspension or any other dosage form. A dosage unit can be measured in a certain volume or weight.

A “single dose dispenser” is a liquid dispenser having a single volume indicator.

The system provides for standardized dosage units for one or more medications based on the use of patient classification codes. The patient classification codes are based upon one or more dosing parameters. A preferred dosing parameter is weight but it can be any other parameter that can be correlated with a recommended dosage amount of the one or more medications. Other examples of dosing parameters include age, sex, height, body surface area, lean body mass, and combinations thereof. Each medication can be tied to a different dosing parameter or set of parameters.

In one preferred embodiment, up to eleven dosing classification codes are provided, based upon the weight ranges shown in the following table:

| Dosing Classification | Weight | Approximate age | Dosage |
|---|---|---|---|
| Code | range (pounds) | (years) | unit (ml) |
| 20 | 19 to <23 | 1 | 2.0 |
| 25 | 23 to <29 | 2 | 3.0 |
| 30 | 29 to <33 | 3 | 3.5 |
| 35 | 33 to <37 | 4 | 4.0 |
| 40 | 37 to <42 | 5 | 4.5 |
| 50 | 42 to <56 | 6 to ≦8 | 5.0 |
| 60 | 56 to <70 | 8 to ≦10 | 7.5 |
| 80 | 70 to <88 | 10 to ≦12 | 10.0 |
| 100 | 88 to <112 | 12 to ≦14 | 12.5 |
| 125 | 112 to <147 | 14 to ≦18 | 15.0 |
| 150 | 147 and above | 18 and older | 20.0 |

Thus, in this embodiment, a code 40 child would receive 4.5 ml of any medication included in the system. A code 80 child would receive 10.0 ml of any medication. A dosing kit for the code 40 child would include a single dose dispenser for 4.5 ml and a code 80 dosing kit would include a single dose dispenser for 10.0 ml.

In this embodiment, a complete line for each medication would desirably have 11 different kits and stock keeping units (SKUs). Each kit would desirably include a calibrated single dose dispenser such as an oral dispensing device (an oral syringe or cup), and a bottle containing sufficient medication to fulfill a complete dosing regiment for the patient. Each kit would further desirably contain a patient information sheet.

The one or more medications can be selected from any therapeutic category, including but not limited to antihistamines, cough suppressants, decongestants, antibiotics, antivirals, anti-infectives, cancer drugs, cardiovascular agents, gastrointestinal drugs, hormones, steroids, neurological drugs, anti-inflammatory drugs, analgesics, psychiatric drugs, respiratory drugs, vitamin supplements, and medical foods. The only requirement for the medication is that it must be able to be formulated in the dosage unit so that the patient receives the recommended dosage of the medication. It may be that a particular medication is not formulatable for each patient classification code and thus cannot be offered for that classification code.

Dosage units may be in any dosage form including liquid, semi-solid, or solid. It is preferable to supply the dosage units in single dose receptacles, or to provide a single dose receptacle with the system. For example, a single dose receptacle may be an oral dispensing device such as an oral syringe or dosing cup for the liquid and semi-solid dosage forms or a blister card for the solid dosage units. Preferably, the single dose receptacle is marked with a single line or otherwise clearly shows the level to which it should be filled for the single dosage unit.

In a preferred embodiment, the single dose dispenser is an oral syringe having a single gradation and the medication is supplied in a bottle to which a press in bottle adaptor (PIBA) can be fitted. The PIBA prevents accidental ingestion of the bottle contents. The oral syringe can be fitted onto the PIBA for filling.

In one embodiment, the invention is for use by physicians and pharmacists as an aid in writing prescriptions for the medications included in the system. If the physician decides to prescribe a certain medication for the patient, he or she matches the patient to the dosing classification code based upon the correlation of the patient's classification parameter with the dosing classification code. The physician then prescribes the medication by the dosing classification code. When the pharmacist dispenses the medication, all he or she must do is select the proper kit for the classification code. The parent then uses the single dose dispenser to administer the medication. The potential for error is reduced at each step.

A dosing kit for a particular medication might provide for sequential dosing requirements, for example if a tapering dosage is needed. In this event, the dosage unit might stay the same and the concentration of the medication change. Alternatively, the dosage unit could change and the medication concentration stays the same. For example, a dosing kit could include a bottle of medication at a particular concentration, a single dose dispenser for 2 ml, and a single dose dispenser for 4 ml, along with patient instructions.

In another embodiment of the dosing system, several patient classification codes are combined into a secondary classification code. This embodiment is practical when the dosing requirements for the medication are less restrictive. Thus the classification codes could be as follows:

| Secondary Dosing | Weight range | Approximate age | Dosage unit |
|---|---|---|---|
| Classification Code | (pounds) | (years) | (ml) |
| 25/40 | 23 to <42 | 2-5 | 2.5 |
| 50 | 42 to <88 | 6-12 | 5.0 |
| 100 | 88 and above | 12 and above | 10.0 |

In an alternate embodiment, a dosing kit includes the medication packaged in bulk and two or more single dose dispensers. This embodiment can be combined with the embodiment having combined classification codes. A dosing kit could be supplied with the medication in bulk, an oral syringe with a single marked line for 2.5 ml, a second oral syringe with a single marked line for 5.0 ml, and a cup with a single marked line for 10.0 ml.

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric. The examples are not intended to restrict the scope of the invention.

### EXAMPLE 1

### Liquid Suspension of Codeine and Chlorpheniramine

The following is a description of one embodiment of the invention which is a suspension for oral administration that contains 0.2 mg chlorpheniramine maleate, an antihistamine, and 1 mg codeine phosphate, a cough suppressant, per ml suspension. The suspension is manufactured according to cGMPs and as known in the art, for example as taught in U.S. Pat. No. 6,869,618 to Kiel et al. The dosing classification code is based upon weight according to the following chart. The chart is developed from the professional labeling section of the OTC Monograph, 21 CFR 341.90, and provides additional dosing information to the physician. This chart represents the Physician's Dosing Guide which will be provided as additional labeling information in the system.

| Dosing | | | Codeine | | |
|---|---|---|---|---|---|
| Classification | Weight range | Approximate | QID dose | Chlorpheniramine | Dosage |
| Code | (pounds) | age (years) | (mg) | QID dose (mg) | unit (ml) |
| 25 | 23 to <29 | 2 | 3.0 | 0.6 | 3.0 |
| 30 | 29 to <33 | 3 | 3.5 | 0.7 | 3.5 |
| 35 | 33 to <37 | 4 | 4.0 | 0.8 | 4.0 |
| 40 | 37 to <42 | 5 | 4.5 | 0.9 | 4.5 |
| 50 | 42 to <56 | 6 to ≦8 | 5.0 | 1.0 | 5.0 |
| 60 | 56 to <70 | 8 to ≦10 | 7.5 | 1.5 | 7.5 |
| 80 | 70 to <88 | 10 to ≦12 | 10.0 | 2.0 | 10.0 |
| 100 | 88 to <112 | 12 to ≦14 | 12.5 | 2.5 | 12.5 |
| 125 | 112 to <147 | 14 to ≦18 | 15.0 | 3.0 | 15.0 |
| 150 | 147 and above | 18 and older | 20.0 | 4.0 | 20.0 |

The kit includes a plurality of dosage units in a 4 oz. bottle. The kit further includes a single marked oral syringe having a single marking for the required dosage unit for the specific classification code. In addition, a physician's professional labeling dosing guide providing dosing information based on weight per 21 CFR 341.90 is also enclosed.

The suspension will be taken every 4 to 6 hours, but not more than 6 doses in 24 hours, or as directed by a doctor.

### EXAMPLE 2

### Liquid Suspension of Albumin Tannate

The following is a description of a second embodiment of the invention which is a suspension for oral administration that contains 125 mg dried egg albumin and 125 mg tannic acid per ml suspension. The suspension is manufactured according to cGMPs and as known in the art, for example as taught in U.S. patent application Ser. No. 11/897,156 to Kiel et al.

| Dosing | | Approximate | Albumin and | Dosage |
|---|---|---|---|---|
| Classification | Weight range | age | tannic acid | unit |
| Code | (pounds) | (years) | (each) (mg) | (ml) |
| 25 | 23 to <29 | 2 | 375 | 3.0 |
| 30 | 29 to <33 | 3 | 437.5 | 3.5 |
| 35 | 33 to <37 | 4 | 500 | 4.0 |
| 40 | 37 to <42 | 5 | 562.5 | 4.5 |
| 50 | 42 to <56 | 6 to ≦8 | 625 | 5.0 |
| 60 | 56 to <70 | 8 to ≦10 | 937.5 | 7.5 |
| 80 | 70 to <88 | 10 to ≦12 | 1250 | 10.0 |
| 100 | 88 to <112 | 12 to ≦14 | 1562.5 | 12.5 |
| 125 | 112 to <147 | 14 to ≦18 | 1875 | 15.0 |
| 150 | 147 and above | 18 and older | 2500 | 20.0 |

### EXAMPLE 3

### Solid Tablets

The following table illustrates the dosage units for a 0.003472 mg/ml blend of triolidone HCl or a 5 weight percent blend of pseudophedrine HCl. The table shows that the same size dosage unit can be used for each medication.

| | | | Tripolidine | Pseudophedrine | |
|---|---|---|---|---|---|
| Dosing | Weight | | HCl | HCl | Dosage |
| Classification | range | Approximate | recommended | recommended | Unit |
| Code | (pounds) | age (years) | dosage (mg) | dosage (mg) | (mg) |
| 15 | 11 to <17.5 | 4 to 12 | 0.1565 | | 45 |
| | | months | | | |
| 20 | 17.5 to <23 | 1 | 0.313 | 4.5 | 90 |
| 25 | 23 to <29 | 2 | 0.625 | 9.0 | 180 |
| 30 | 29 to <33 | 3 | 0.7294 | 10.5 | 210 |
| 35 | 33 to <37 | 4 | 0.8336 | 12.0 | 240 |
| 40 | 37 to <42 | 5 | 0.938 | 13.5 | 270 |
| 50 | 42 to <56 | 6 to ≦8 | 1.042 | 15 | 300 |
| 60 | 56 to <70 | 8 to ≦10 | 1.146 | 22.5 | 450 |
| 80 | 70 to <88 | 10 to ≦12 | 1.25 | 30 | 600 |
| 100 | 88 to <112 | 12 to ≦14 | 1.5625 | 37.5 | 750 |
| 125 | 112 to <147 | 14 to ≦18 | 1.875 | 45 | 900 |
| 150 | 147 and | 18 and older | 2.5 | 60 | 1200 |
| | above | | | | |

### EXAMPLE 4

### Other Liquid Formulations

To fit the above dosing requirements, the following formulations can be used for other liquid medications.

| Medication | Formulation (mg/ml) |
|---|---|
| Pyrilamine maleate | 2.5 |
| Diphenhydramine HCl | 2.5 |
| Dextromethoraphan | 1.5 |
| Pseudophedrine | 3.0 |
| Phenylephrine | 0.5 |
| Guaifenesin | 10.0 |
| Doxylamine succinate | 0.625 |
| Brompheniramine | 0.2 |

It will be appreciated that the dosing system of the present invention may be used for other medications.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

1. A medication dosing system for one or more medications based on patient classification codes and standardized dosage units, comprising:
one or more patient classification codes correlated with one or more patient classification parameters;
a dosage unit for each classification code;
wherein the dosage unit is the same for each classification code for each medication; and
wherein each medication is formulated so that the patient receives the recommended dosage amount of the medication.

2. The medication dosing system of claim 1 wherein the medication is packaged in individual dosage units.

3. The medication dosing system of claim 1 wherein the medication is packaged in bulk and the system further includes a single dose dispenser for each dosage unit.

4. The medication dosing system of claim 1 wherein the single dose dispenser is an oral syringe having a single gradation.

5. The medication dosing system of claim 1 wherein the single dose dispenser is a dosing cup having a single gradation.

6. The medication dosing system of claim 1, wherein there are from one to twenty patient classification codes.

7. The medication dosing system of claim 1 wherein the classification parameter is selected from one or more of weight, height, age, sex, and body surface area.

8. The medication dosing system of claim 1 wherein a dosage unit corresponds to more than one classification codes.

9. A medication dosing kit for a patient meeting one or more classification parameters and assigned a classification code based upon the one or more classification parameters, comprising:
the medication packaged in single dosage units or in bulk;
a single dose dispenser if the medication is packaged in bulk, wherein the single dose dispenser is designed to deliver a single dosage unit; and
wherein the dosage unit is unique for the patient classification code.

10. The medication dosing kit of claim 9 further comprising a physician information sheet for the medication detailing the patient classification codes, the classification parameters, and the quantities of medication in each dosage unit.

11. The medication dosing kit of claim 9 further comprising a patient information sheet for the medication.

12. The medication dosing kit of claim 9 wherein the single dose dispenser is an oral syringe or a dosing cup having a single gradation.

13. The medication dosing kit of claim 9 wherein the classification parameter is selected from one or more of weight, height, age, sex, and body surface area.

14. The medication dosing kit of claim 9 wherein one or more classification codes are combined into a secondary classification code and the dosage unit corresponds to the secondary classification code.

15. A method for prescribing, dispensing, and administering a medication to a patient, comprising the steps:
deciding to prescribe a medication to a patient;
determining a classification code for the patient based upon patient classification parameters;
dispensing the medication to the patient by selecting a medication dosing kit specific for the classification code, wherein the medication dosing kit comprises the medication packaged in single dosage units or in bulk and a single dose dispenser if the medication is packaged in bulk; and
administering the medication in single dosage units or using the single dose dispenser.

16. The method of claim 15, wherein a physician performs the steps of deciding to prescribe and determining a classification code and a pharmacist performs the step of dispensing the medication.

17. The method of claim 16, further comprising the step of the physician writing a prescription for the medication using the classification code.

18. The method of claim 15, wherein the patient classification parameter is selected from one or more of weight, height, age, sex, and body surface area.

19. The method of claim 15, wherein the medication is packaged in bulk and the single dose dispenser is an oral syringe or a dosing cup having a single gradation.
